(19) United States
(12) Patent Application Publication
(10) Pub. No.: US 2015/0202196 A1
(43) Pub. Date: Jul. 23, 2015

(54) Methods of Using C-Met Modulators

(71) Applicant: Exelixis, Inc., South San Francisco, CA (US)

(72) Inventors: Lynne Canne Bannen, New Castle, IN (US); Diva Sze-Ming Chan, Oakland, CA (US); Timothy Patrick Forsyth, Hayward, CA (US); Richard George Khoury, San Mateo, CA (US); James William Leahy, Tampa, FL (US); Morrison B. Mac, San Francisco, CA (US); Larry W. Mann, Richland, MI (US); John M. Nuss, Danville, CA (US); Jason Jevious Parks, Elk Grove, CA (US); Yong Wang, Foster City, CA (US); Wei Xu, Danville, CA (US); Diane Simeone, Ann Arbor, MI (US)

(21) Appl. No.: 14/518,733

(22) Filed: Oct. 20, 2014

Related U.S. Application Data
(63) Continuation of application No. 13/087,094, filed on Apr. 14, 2011, now abandoned.
(60) Provisional application No. 61/325,095, filed on Apr. 16, 2010.

(51) Int. Cl. A61K 31/47; A61N 5/10; A61K 31/7068

(52) U.S. Cl. CPC A61K 31/47; A61K 31/7068; A61N 5/10

(57) ABSTRACT

Disclosed are methods of treating cancer by administering a compound of Formula I,

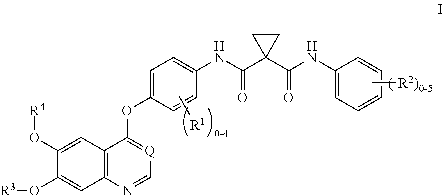

or a pharmaceutically acceptable salt thereof, in combination with gemcitabine (GEM), or a pharmaceutically acceptable salt thereof, and optionally one or more additional treatments,
wherein:
R¹ is halo;
R² is halo;
R³ is (C₁-C₆)alkyl;
R⁴ is (C₁-C₆)alkyl; and
Q is CH or N.

### CROSS REFERENCE TO RELATED APPLICATIONS

This application claims the benefit of U.S. Non-Provisional patent application Ser. No. 13/087,094, filed Apr. 14, 2011, which claims the benefit of U.S. Provisional Patent Application Ser. No. 61/325,095, filed Apr. 16, 2010, which is incorporated herein by reference in its entirety.

### FIELD OF THE INVENTION

This invention relates to methods of using c-Met modulators, and specifically c-Met modulators in combination with other anti-cancer agents and/or radiation, which can be useful for the modulation of various cellular activities and for the treatment of various diseases as described in the specification.

### BACKGROUND OF THE INVENTION

Traditionally, dramatic improvements in the treatment of cancer are associated with identification of therapeutic agents acting through novel mechanisms. One mechanism that can be exploited in cancer treatment is the modulation of protein kinase activity because signal transduction through protein kinase activation is responsible for many of the characteristics of tumor cells. Protein kinase signal transduction is of particular relevance in, for example, thyroid, gastric, head and neck, lung, breast, prostate, and colorectal cancers, as well as in the growth and proliferation of brain tumor cells.

Protein kinases can be categorized as receptor type or non-receptor type. Receptor-type tyrosine kinases are comprised of a large number of transmembrane receptors with diverse biological activity. For a detailed discussion of the receptor-type tyrosine kinases, see Plowman et al., DN&P 7(6): 334-339, 1994. Since protein kinases and their ligands play critical roles in various cellular activities, deregulation of protein kinase enzymatic activity can lead to altered cellular properties, such as the uncontrolled cell growth associated with cancer. In addition to oncological indications, altered kinase signaling is implicated in numerous other pathological diseases, including, for example, immunological disorders, cardiovascular diseases, inflammatory diseases, and degenerative diseases. Therefore, protein kinases are attractive targets for small molecule drug discovery. Particularly attractive targets for small-molecule modulation with respect to antiangiogenic and antiproliferative activity include receptor type tyrosine kinases Ret, c-Met, and VEGFR2.

The kinase c-Met is the prototypic member of a subfamily of heterodimeric receptor tyrosine kinases (RTKs) that includes Met, Ron, and Sea. The endogenous ligand for c-Met is the hepatocyte growth factor (HGF), a potent inducer of angiogenesis. Binding of HGF to c-Met induces activation of the receptor via autophosphorylation, resulting in an increase of receptor dependent signaling, which promotes cell growth and invasion. Anti-HGF antibodies or HGF antagonists have been shown to inhibit tumor metastasis in vivo (See: Maulik et al Cytokine & Growth Factor Reviews 2002 13, 41-59). c-Met, VEGFR2, and/or Ret overexpression has been demonstrated on a wide variety of tumor types, including breast, colon, renal, lung, squamous cell myeloid leukemia, hemangiomas, melanomas, astrocytomas, and glioblastomas. The Ret protein is a transmembrane receptor with tyrosine kinase activity. Ret is mutated in most familial forms of medullary thyroid cancer. These mutations activate the kinase function of Ret and convert it into an oncogene product.

Inhibition of EGF, VEGF, and ephrin signal transduction will prevent cell proliferation and angiogenesis, two key cellular processes needed for tumor growth and survival (Matter A. Drug Disc. Technol. 2001 6, 1005-1024). Kinase KDR (kinase insert domain receptor tyrosine kinase) and flt-4 (fins-like tyrosine kinase-4) are both vascular endothelial growth factor (VEGF) receptors. Inhibition of EGF, VEGF, and ephrin signal transduction will prevent cell proliferation and angiogenesis, two key cellular processes needed for tumor growth and survival (Matter A. Drug Disc. Technol. 2001 6, 1005-1024). EGF and VEGF receptors are desirable targets for small molecule inhibition.

Accordingly, small-molecule compounds that specifically inhibit, regulate, and/or modulate the signal transduction of kinases, specificatially the Ret, c-Met, and VEGFR2 kinases described above, are particularly desirable as a means to treat or prevent disease states associated with abnormal cell proliferation and angiogenesis. One such small-molecule is N-(4-{[6,7-bis(methyloxy)quinolin-4-yl]oxy}phenyl)-N′-(4-fluorophenyl)cyclopropane-1,1-dicarboxamide (Compound 1), which has the chemical structure:

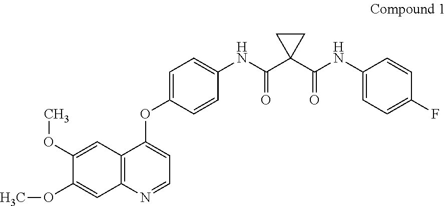 [SMILES: COc1cc2nccc(Oc3ccc(NC(=O)C4(C(=O)Nc5ccc(F)cc5)CC4)cc3)c2cc1OC]

WO 2005/030140 describes the synthesis of N-(4-{[6,7-bis(methyloxy)quinolin-4-yl]oxy}phenyl)-M-(4-fluorophenyl)cyclopropane-1,1-dicarboxamide (Examples 25, 37, 38, and 48) and discloses the therapeutic activity of this molecule to inhibit, regulate, and/or modulate the signal transduction of kinases (Assays, Table 4, entry 289). Compound 1 has been measured to have a c-Met IC₅₀ value of 1.3 nanomolar (nM) and a Ret IC₅₀ value of 5.2 nanomolar (nM).

Finding new methods for using Compound 1 in combination therapies for treating disease is desirable.

### SUMMARY OF THE INVENTION

The summary of the invention only summarizes certain aspects of the invention and is not intended to be limiting in nature. These aspects and other aspects and embodiments are described more fully below. In the event of a discrepancy between the express disclosure of this specification and the references incorporated by herein reference, the express disclosure of this specification shall control.

One aspect of this invention relates to methods of treating diseases comprising administering to a patient in need of the treatment a compound of Formula I as defined in the Detailed Description of the Invention, in combination with gemcitabine (GEM), with optionally one or more additional treatment(s).

### BRIEF DESCRIPTION OF THE FIGURES

FIG. 1 shows the experimental XRPD pattern for crystalline Compound I at 25° C.

FIG. 2 shows the solid state ¹³C NMR spectrum of crystalline Compound I.

FIG. 3 shows the solid state ¹⁵N NMR spectrum of crystalline Compound I.

FIG. 4 shows the solid state ¹⁹F NMR spectrum of crystalline Compound I.

FIG. 5 shows the thermal gravimetric analysis (TGA) of crystalline Compound I.

FIG. 6 shows the differential scanning calorimetry (DSC) of crystalline Compound I.

FIG. 7 shows the moisture sorption of crystalline Compound I.

FIG. 8A shows the effect of c-Met inhibition with and without Gemcitabine on subcutaneous primary human pancreatic cancer growth in NOD/SCID mice.

FIG. 8B shows the average tumor size±SEM from three separate tumors.

FIG. 8C the CSC percentage in different treatment groups.

FIG. 9A shows the effect of Compound 1 and Gemcitabine on orthotopic tumor growth in xenograft mice.

FIG. 9B summarizes the results the experiments depicted in FIG. 9A.

### DETAILED DESCRIPTION OF THE INVENTION

### Abbreviations and Definitions

The following abbreviations and terms have the indicated meanings throughout:

| Abbreviation | Meaning |
|---|---|
| Ac | Acetyl |
| Br | Broad |
| ° C. | Degrees Celsius |
| c- | Cyclo |
| CBZ | CarboBenZoxy = benzyloxycarbonyl |
| d | Doublet |
| dd | Doublet of doublet |
| dt | Doublet of triplet |
| DCM | Dichloromethane |
| DME | 1,2-dimethoxyethane |
| DMF | N,N-dimethylformamide |
| DMSO | Dimethyl sulfoxide |
| Dppf | 1,1′-bis(diphenylphosphano)ferrocene |
| EI | Electron Impact ionization |
| G | Gram(s) |
| h or hr | Hour(s) |
| HPLC | High pressure liquid chromatography |
| L | Liter(s) |
| M | Molar or molarity |
| m | Multiplet |
| Mg | Milligram(s) |
| MHz | Megahertz (frequency) |
| Min | Minute(s) |
| mL | Milliliter(s) |
| μL | Microliter(s) |
| μM | Micromole(s) or micromolar |
| mM | Millimolar |
| Mmol | Millimole(s) |
| Mol | Mole(s) |
| MS | Mass spectral analysis |
| N | Normal or normality |
| nM | Nanomolar |
| NMR | Nuclear magnetic resonance spectroscopy |
| q | Quartet |
| RT | Room temperature |
| s | Singlet |
| t or tr | Triplet |
| TFA | Trifluoroacetic acid |
| THF | Tetrahydrofuran |
| TLC | Thin layer chromatography |

The symbol “-” indicates a single bond, and “=” indicates a double bond.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes, a particular atom in a structure is described in textual formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to the description of otherwise complex structures.

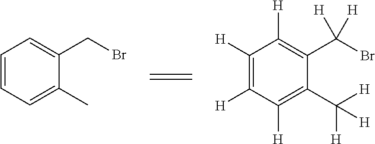

If a group “R” is depicted as “floating” on a ring system, for example in the formula:

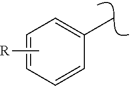

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused ring system, as for example in the formulae:

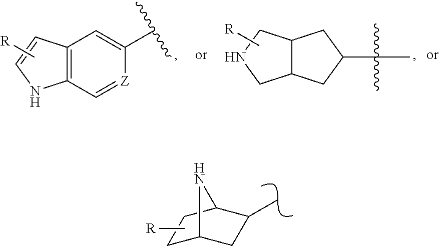

then, unless otherwise defined, a substituent “R” may reside on any atom of the fused ring system, assuming replacement of a depicted hydrogen (for example the —NH— in the formula above), implied hydrogen (for example as in the formula above, where the hydrogens are not shown but understood to be present), or expressly defined hydrogen (for example where in the formula above, “Z” equals ═CH—) from one of the ring atoms, so long as a stable structure is formed. In the example depicted, the “R” group may reside on either the 5-membered or the 6-membered ring of the fused ring system.

When a group “R” is depicted as existing on a ring system containing saturated carbons, such as in the formula:

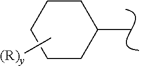

where, in this example, “y” can be more than one, assuming each replaces a currently depicted, implied, or expressly defined hydrogen on the ring; then, unless otherwise defined, where the resulting structure is stable, two “R's” may reside on the same carbon. A simple example is when R is a methyl group; there can exist a geminal dimethyl on a carbon of the depicted ring (an “annular” carbon). In another example, two R's on the same carbon, including that carbon, may form a ring, thus creating a spirocyclic ring (a “spirocyclyl” group) structure with the depicted ring as for example in the formula:

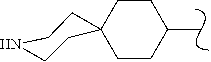

As used herein, “halogen” or “halo” refers to fluorine, chlorine, bromine, or iodine.

As used herein, “yield” for each of the reactions is expressed as a percentage of the theoretical yield.

As used herein, “hormone therapy” or “hormonal therapy” includes, for example, treatment with one or more of the following: steroids (e.g. dexamethasone), finasteride, tamoxifen, and an aromatase inhibitor.

As used herein, “patient” includes humans and other animals, particularly mammals, and other organisms. The methods are thus applicable to both human therapy and veterinary applications. In one embodiment, the patient is a mammal, and in another embodiment, the patient is human.

As used herein, a “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, and in S. M. Berge, et al., “Pharmaceutical Salts,” J. Pharm. Sci., 1977; 66:1-19, both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, malic acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, salicylic acid, and the like.

As used herein, “platin(s),” and “platin-containing agent(s)” include, for example, cisplatin, carboplatin, and oxaliplatin.

As used herein, “prodrug” refers to compounds that are transformed (typically rapidly) in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example, with about one to about six carbons) wherein the alkyl group is a straight or branched chain. Acceptable esters also include cycloalkyl esters and arylalkyl esters such as, but not limited to, benzyl. Examples of pharmaceutically acceptable amides of the compounds of this invention include, but are not limited to, primary amides, and secondary and tertiary alkyl amides (for example, with about one to about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference.

As used herein, “taxane(s)” includes, for example, one or more of the following: Paclitaxel (Taxol®) and Docetaxel (Taxoter®).

As used herein, a “therapeutically effective amount” is an amount of a compound of the invention, that when administered to a patient, ameliorates a symptom of the disease. A therapeutically effective amount is intended to include an amount of a compound alone or in combination with other active ingredients effective to modulate Ret, c-Met, and/or VEGFR2, or effective to treat or prevent cancer. The amount of a compound of the invention which constitutes a “therapeutically effective amount” will vary depending on the compound, the disease state and its severity, the age of the patient to be treated, and the like. The therapeutically effective amount can be determined by one of ordinary skill in the art having regard to their knowledge and to this disclosure.

As used herein, “topoisomerase inhibitor” includes, for example, one or more of the following: amsacrine, camptothecin, etoposide, etoposide phosphate, exatecan, irinotecan, lurtotecan, teniposide, and topotecan.

As used herein, “treating” or “treatment” of a disease, disorder, or syndrome includes (i) preventing the disease, disorder, or syndrome from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction, and the severity of the condition may be necessary, and these necessary adjustments will be ascertainable with routine experimentation by one of ordinary skill in the art.

As used herein, “amorphous” refers to a solid form of a molecule and/or ion that is not crystalline. An amorphous solid does not display a definitive X-ray diffraction pattern with sharp maxima.

As used herein, the term “substantially pure” indicates that the referenced crystalline form of the (L)-malate salt form of Compound 1 contains at least about 90 weight percent based on the weight of such crystalline form. The term “at least about 90 weight percent,” while not intending to limit the applicability of the doctrine of equivalents to the scope of the claims, includes, but is not limited to, about 90, about 91, about 92, about 93, about 94, about 95, about 96, about 97, about 98, about 99, and about 100 weight percent, based on the weight of the referenced crystalline form. The remainder may comprise another form(s) of the (L)-malate salt form of Compound 1, reaction impurities, and/or processing impurities that arise, for example, when the crystalline form is prepared. The presence of reaction impurities and/or processing impurities may be determined by analytical techniques known in the art, such as, for example, chromatography, nuclear magnetic resonance spectroscopy, mass spectroscopy, and/or infrared spectroscopy.

It is to be appreciated that certain features of the invention that are, for clarity reasons, described above and below in the context of separate embodiments, may also be combined to form a single embodiment. Conversely, various features of this disclosure that are, for brevity reasons, described in the context of a single embodiment, may also be combined so as to form sub-combinations thereof. The disclosure is further illustrated by the following examples, which are not to be construed as limiting the disclosure in scope or spirit to the specific procedures described in them.

The definitions set forth herein take precedence over definitions set forth in any patent, patent application, and/or patent application publication incorporated herein by reference. All measurements are subject to experimental error and are within the spirit of the invention.

### Aspects and Embodiments of the Invention

The invention relates to a method of treating a disease, comprising administering to a patient in need of such treatment a compound of Formula I:

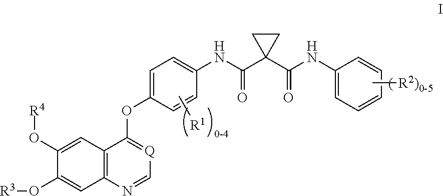

or a pharmaceutically acceptable salt thereof, in combination with gemcitabine (GEM), or a pharmaceutically acceptable salt thereof, and optionally one or more additional treatments,
wherein:
R¹ is halo;
R² is halo;
R³ is (C₁-C₆)alkyl;
R⁴ is (C₁-C₆)alkyl; and

### Q is CH or N.

Gemcitabine (GEM) has the following structure:

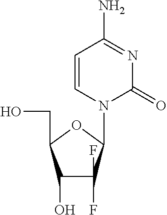

and can be in the form of a pharmaceutically acceptable salt, such as an acid addition salt. One such example of an acid addition salt of gemcitabine that can be used is gemcitabine hydrochloride. One form of gemcitabine is currently available as GEMZAR®.

In one embodiment, the compound of Formula I is the compound of Formula I(a):

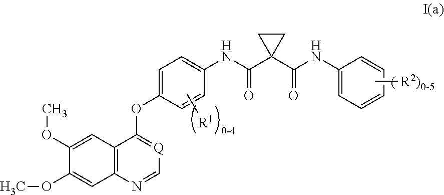

or a pharmaceutically acceptable salt thereof, wherein:
R¹ is halo;
R² is halo; and

### Q is CH or N.

In another embodiment, the Compound of Formula I is Compound 1:

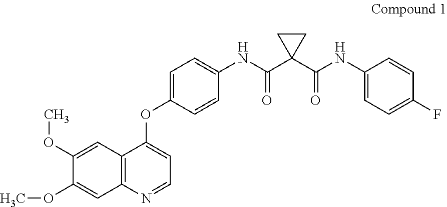 [SMILES: COc1cc2nccc(Oc3ccc(NC(=O)C4(C(=O)Nc5ccc(F)cc5)CC4)cc3)c2cc1OC]

or a pharmaceutically acceptable salt thereof.

In another embodiment, the compound of Formula I, I(a), or Compound 1, or a pharmaceutically acceptable salt thereof, is administered as a pharmaceutical composition, wherein the pharmaceutical composition additionalyy comprises a pharmaceutically acceptable carrier, excipient, or diluent.

In another embodiment, the invention optionally comprises one or more additional treatment(s). The one or more additional treatment(s) are selected from the group consisting of (1) surgery, (2) one or more additional chemotherapeutic agent(s), (3) one or more hormone therapy(ies), (4) one or more antibody(ies), and (5) one or more immunotherapy(ies), (6) radioactive iodine therapy, and (7) radiation.

The compound of Formula I, I(a), and Compound 1, and all embodiments as described herein, includes the recited compounds, as well as individual isomers and mixtures of isomers. In each instance, the compound of Formula I, I(a), and Compound 1 includes the pharmaceutically acceptable salts, hydrates, and/or solvates of the recited compounds and any individual isomers or mixture of isomers thereof.

In another embodiment, the compound of Formula I can be the malate salt of the compound of Formula I.

In another embodiment, the compound of Formula I can be the (L)-malate salt of the compound of Formula I.

In another embodiment, the compound of Formula I can be the (D)-malate salt of the compound of Formula (I).

In another embodiment, the compound of Formula I can be malate salt of the compound of Formula I(a).

In another embodiment, the compound of Formula I can be the (L)-malate salt of the compound of Formula I(a).

In another embodiment, the compound of Formula I can be (D)-malate salt of the compound of Formula I(a).

In another embodiment, the compound of Formula I can be the malate salt of Compound 1.

In another embodiment, the compound of Formula I can be the (L)-malate salt of Compound 1, which has the following structure.

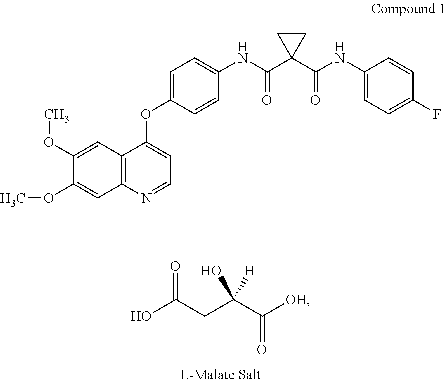 [SMILES: COc1cc2nccc(Oc3ccc(NC(=O)C4(C(=O)Nc5ccc(F)cc5)CC4)cc3)c2cc1OC ; O=C(O)C[C@@H](O)C(=O)O]

In another embodiment, the compound of Formula I can be the (D)-malate salt of Compound 1.

In one embodiment, the disease being treated is cancer.

In another embodiment, the disease being treated is selected from leukemia, breast cancer, brain cancer, lung cancer (including non-small cell lung cancer), multiple myeloma, prostate cancer, colon cancer, head and neck cancer, medullary thyroid cancer, pancreatic cancer, and melanoma.

In another embodiment, the disease being treated is pancreatic cancer.

Other non-limiting examples of additional treatments that can be used in the methods described herein include anti-cancer or chemotherapeutic agent(s). Non-limiting examples of anti-cancer agents include rapamycin, a rapamycin analogue, an alkylating agent(s), a taxane(s), and a platin(s). Non-limiting examples of chemotherapeutic agent(s) include rapamycin, temozolomide, paclitaxel, docetaxel, carboplatin, cisplatin, oxaliplatin, gefitinib (Iressa®), erlotinib (Tarceva®), Zactima (ZD6474), HKI-272, pelitinib, canertinib, and lapatinib.

In another embodiment, the one or more additional treatment(s) is one or more hormone therapy(ies). Non-limiting examples of the hormone therapy(ies) that can be used in this embodiment include tamoxifen, Toremifene (Fareston), Fulvestrant (Faslodex), Megestrol acetate (Megace), ovarian ablation, Raloxifene, a luteinizing hormone-releasing hormone (LHRH) analog (including goserelin and leuprolide), Megestrol acetate (Megace), and one or more aromatase inhibitor(s). In another embodiment, one or more of the aromatase inhibitor(s) is selected from letrozole (Femara), anastrozole (Arimidex), and exemestane (Aromasin). In another embodiment, one or more of the hormone therapy(ies) is selected from tamoxifen and an aromatase inhibitor.

Non-limiting examples of the radiation treatment that can be used in this embodiment include external beam radiation, interstitial radiotherapy, and stereotactic radiosurgery. Non-limiting examples of the additional chemotherapeutic agent(s) that can be used in this embodiment include carmustine (BCNU), Erlotinib (Tarceva), bevacizumab, gefitinib (Iressa), rapamycin, cisplatin, BCNU, lomustine, procarbazine, and vincristine. A non-limiting example of the antiseizure agent(s) that can be used in this embodiment is diphenylhydantoin (Dilantin). A non-limiting example of the agent that can be used to reduce swelling in this embodiment is dexamethasone (Decadron).

In another embodiment, the one or more additional treatments are radiation and surgery.

In another embodiment, the one or more additional treatments are radiation and one or more additional chemotherapeutic agent(s).

In another embodiment, the one or more additional treatments are surgery and one or more additional chemotherapeutic agent(s).

In one embodiment, the disease is pancreatic cancer, and the method further comprises administering radiation therapy and surgery to the patient.

In another embodiment, the compound of Formula I, I(a), or Compound 1 is in the form of a pharmaceutical composition comprising a pharmaceutically acceptable carrier, excipient, or diluent. In this embodiment, GEM can also be in the form of a pharmaceutical composition comprising a pharmaceutically acceptable carrier, excipient, or diluent.

In another embodiment, Compound 1 is a crystalline form of the (L)-malate salt and/or the (D)-malate salt of Compound 1, which includes both crystalline forms of the malate salt of Compound 1, as described herein. As is known in the art, the crystalline (L)-malate salt of Compound 1 will form the same crystalline form and have the same properties as the crystalline (D)-malate salt of Compound 1. See WO 2008/083319, which discusses the properties of crystalline enantiomers. Both crystalline forms of the malate salt of Compound 1, and methods of making and characterizing them, are fully described in PCT/US10/21194, which is incorporated herein by reference in its entirety.

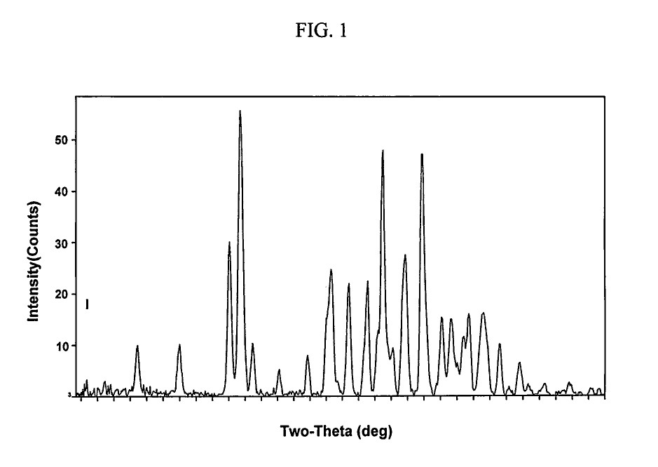

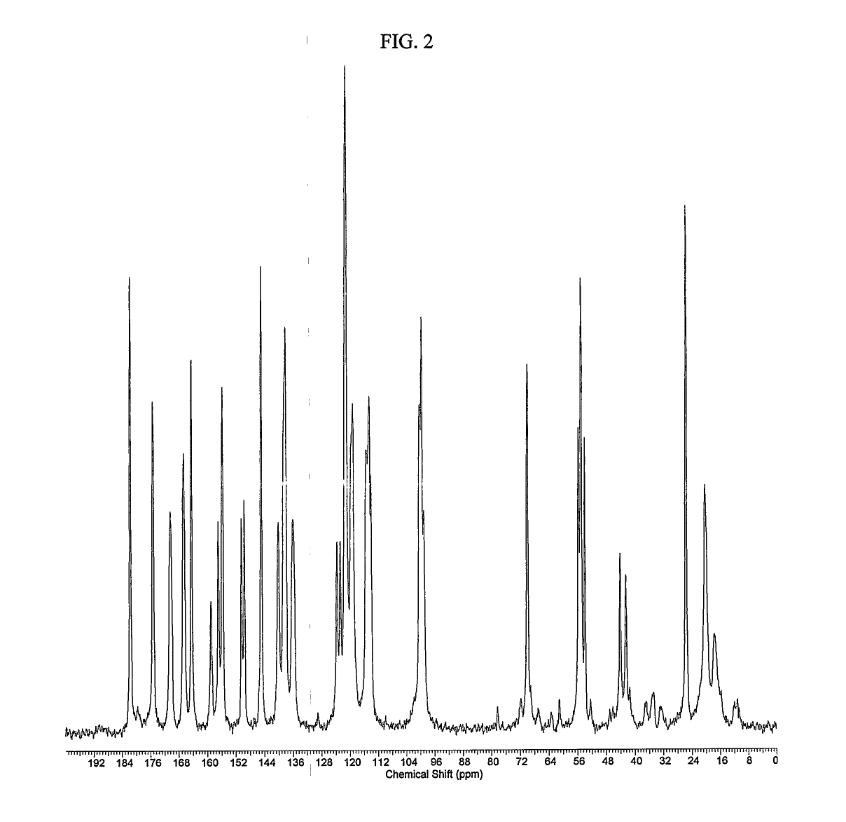

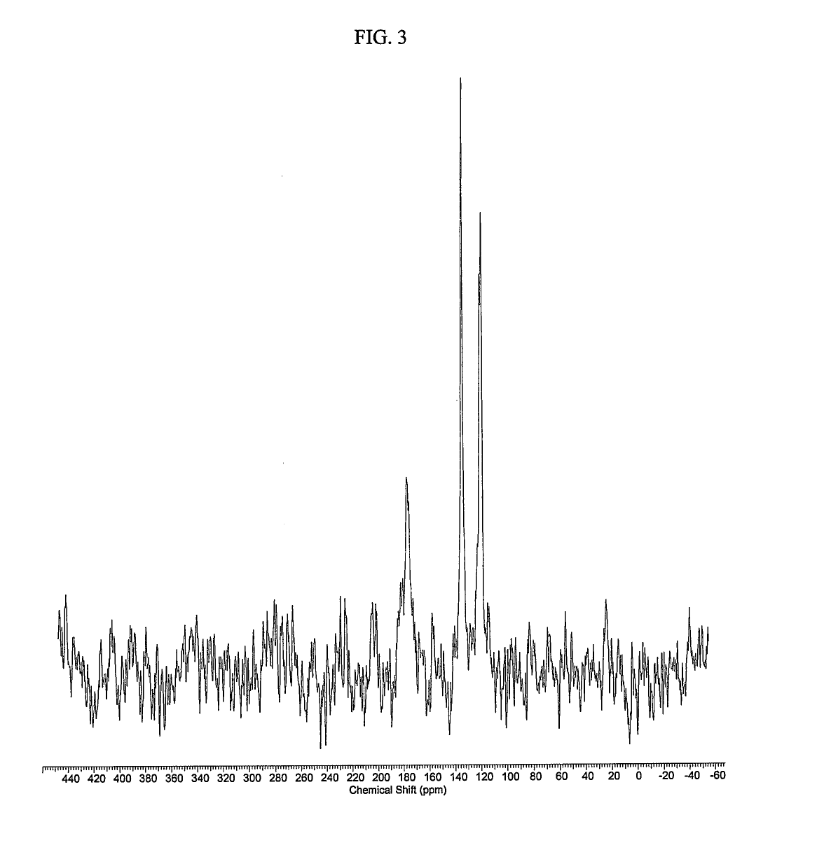

The crystalline form of the (L)-malate salt and/or the (D)-malate salt of Compound 1, as described herein, may be characterized by at least one of the following:
(i) a solid state ¹³C NMR spectrum with peaks at 18.1, 42.9, 44.5, 70.4, 123.2, 156.2, 170.8, 175.7, and 182.1 ppm, ±0.2 ppm;
(ii) a solid state ¹³C NMR spectrum substantially in accordance with the pattern shown in FIG. 2;
(iii) an x-ray powder diffraction pattern (CuKα λ=1.5418 Å) comprising four or more peaks selected from: 6.4, 9.0, 12.0, 12.8, 13.5, 16.9, 19.4, 21.5, 22.8, 25.1, and 27.6° 2θ±0.2° 2θ, wherein measurement of the crystalline form is at an ambient room temperature;
(iv) an x-ray powder diffraction (XRPD) spectrum substantially in accordance with the pattern shown in FIG. 1;
(v) a solid state ¹⁵N NMR spectrum with peaks at 118.6, 119.6, 120.7, 134.8, 167.1, 176.0, and 180 ppm, ±0.2 ppm; and/or
(vi) a solid state ¹⁵N NMR spectrum substantially in accordance with the pattern shown in FIG. 3.

Other solid state properties which may be used to characterize the crystalline N-1 forms of the (L)-malate salt and/or the (D)-malate salt of Compound 1 are shown in the figures and discussed in the examples below.

In other embodiments, the compound of Formula I is a substantially pure crystalline form of the (L)-malate salt and/or the (D)-malate salt of Compound 1.

The crystalline form of the (L)-malate salt and/or the (D)-malate salt of Compound 1 can occur as mixtures. The mixtures may have from greater than zero weight percent to less than 100 weight percent of the (L)-malate salt form and from less than 100 weight percent to greater than zero weight percent (D)-malate salt form, based on the total weight of (L)-malate salt form and (D)-malate salt form. In another embodiment, the mixture comprises from about 1 to about 99 weight percent of the (L)-malate salt form and from about 99 to about 1 weight percent of the (D)-malate salt form, based on the total weight of the (L)-malate salt form and the (D)-malate salt form in said mixture. In a further embodiment, the mixture comprises from about 90 weight percent to less than 100 weight percent (L)-malate salt form and from greater than zero weight percent to about 10 weight percent (D)-malate salt form, based on the total weight of the (L)-malate salt form and the (D)-malate salt form. Accordingly, the mixture may have 1 to 10 percent by weight of the (L)-malate salt form; 11 to 20 percent by weight of the (L)-malate salt form; 21 to 30 percent by weight of the (L)-malate salt form; 31 to 40 percent by weight of the (L)-malate salt form; 41 to 50 percent by weight of the (L)-malate salt form; 51 to 60 percent by weight of the (L)-malate salt form; 61 to 70 percent by weight of the (L)-malate salt form; 71 to 80 percent by weight of the (L)-malate salt form; 81 to 90 percent by weight of the (L)-malate salt form; or 91 to 99 percent by weight of the (L)-malate salt form with the remaining weight percentage of malate salt being that of the (D)-malate salt form.

### General Preparation Methods and Analysis of Crystalline Forms

Crystalline forms may be prepared by a variety of methods including, but not limited to, crystallization or recrystallization from a suitable solvent mixture, sublimation, growth from a melt, solid state transformation from another phase, crystallization from a supercritical fluid, and jet spraying. Techniques for crystallization or recrystallization of crystalline forms of a solvent mixture include, but are not limited to, evaporation of the solvent, decreasing the temperature of the solvent mixture, crystal seeding of a supersaturated solvent mixture of the compound and/or salt thereof, crystal seeding a supersaturated solvent mixture of the compound and/or a salt from thereof, freeze drying the solvent mixture, and adding antisolvents (countersolvents) to the solvent mixture. High throughput crystallization techniques may be employed to prepare crystalline forms including polymorphs.

Crystals of drugs, including polymorphs, their methods of preparation, and the characterization of drug crystals, are discussed in Solid-State Chemistry of Drugs, S. R. Byrn, R. R. Pfeiffer, and J. G. Stowell, 2^{nd} Edition, SSCI, West Lafayette, Ind. (1999).

In a crystallization technique in which a solvent is employed, the solvent is typically chosen based on one or more factors including, but not limited to, solubility of the compound, crystallization technique utilized, and vapor pressure of the solvent. Combinations of solvents may be employed. For example, the compound may be solubilized in a first solvent to afford a solution, followed by the addition of an antisolvent to decrease the solubility of the compound in the solution and precipitate the formation of crystals. An antisolvent is a solvent in which a compound has low solubility.

In one method that can be used in preparing crystals, the (L)-malate salt of Compound 1 can be suspended and/or stirred in a suitable solvent to afford a slurry, which may be heated to promote dissolution. The term “slurry,” as used herein, means a saturated solution of the compound, wherein such solution may contain an additional amount of compound to afford a heterogeneous mixture of compound and solvent at a given temperature.

Seed crystals may be added to any crystallization mixture to promote crystallization. Seeding may be employed to control growth of a particular polymorph and/or to control the particle size distribution of the crystalline product. Accordingly, calculation of the amount of seeds needed depends on the size of the seed available and the desired size of an average product particle as described, for example, in Programmed Cooling Batch Crystallizers,” J. W. Mullin and J. Nyvlt, Chemical Engineering Science, 1971, 26, 3690377. In general, seeds of small size are needed to effectively control the growth of crystals in the batch. Seeds of small size may be generated by sieving, milling, or micronizing large crystals, or by microcrystallizing a solution. In the milling or micronizing of crystals, care should be taken to avoid changing crystallinity from the desired crystalline form (i.e., changing to an amorphous or other polymorphic form).

A cooled crystallization mixture may be filtered under vacuum and the isolated solid product washed with a suitable solvent, such as, for example, cold recrystallization solvent. After washing, the product may be dried under a nitrogen purge to afford the desired crystalline form. The product may be analyzed by a suitable spectroscopic or analytical technique including, but not limited to, differential scanning calorimetry (DSC), x-ray powder diffraction (XRPD), and thermogravimetric analysis (TGA), to ensure that the crystalline form of the compound has been formed. The resulting crystalline form may be produced in an amount greater than about 70 weight percent isolated yield, based on the weight of the compound originally employed in the crystallization procedure, and preferably greater than about 90 weight percent isolated yield. Optionally, the product may be delumped by comilling or passing through a mesh screen.

### Preparation of Crystalline (L)-Malate Salt of Compound 1

The preparation of the captioned salt and its characterization is described in PCT/US10/21194.

### Solid State Nuclear Magnetic Resonance (SSNMR)

All solid-state C-13 NMR measurements were made with a Bruker DSX-400, 400 MHz NMR spectrometer. High resolution spectra were obtained using high-power proton decoupling, the TPPM pulse sequence, and ramp amplitude cross-polarization (RAMP-CP) with magic-angle spinning (MAS) at approximately 12 kHz (A. E. Bennett et al, J. Chem. Phys., 1995, 103, 6951 and G. Metz, X. Wu and S. O. Smith, J. Magn. Reson. A., 1994, 110, 219-227). Approximately 70 mg of sample, packed into a canister-design zirconia rotor, was used for each experiment. Chemical shifts (δ) were referenced to external adamantane with the high frequency resonance being set to 38.56 ppm (W. L. Earl and D. L. VanderHart, J. Magn. Reson., 1982, 48, 35-54).

### (L)-Malate Salt of Compound 1

The solid state ¹³C NMR spectrum of the crystalline (L)-malate salt of Compound 1 is shown in FIG. 2. The entire list of peaks, or a subset thereof, may be sufficient to characterize crystalline (L)-malate salt of Compound 1.

SS ¹³C NMR Peaks: 18.1, 20.6, 26.0, 42.9, 44.5, 54.4, 55.4, 56.1, 70.4, 99.4, 100.1, 100.6, 114.4, 114.9, 115.8, 119.6, 120.1, 121.6, 123.2, 124.1, 136.4, 138.6, 140.6, 145.4, 150.1, 150.9, 156.2, 157.4, 159.4, 164.9, 167.1, 170.8, 175.7, and 182.1 ppm, ±0.2 ppm.

FIG. 3 shows the solid state ¹⁵N NMR spectrum of the crystalline (L)-malate salt of Compound 1. The spectrum shows peaks at 118.6, 119.6, 120.7, 134.8, 167.1, 176.0, and 180 ppm, ±0.2 ppm. The entire list of peaks, or a subset thereof, may be sufficient to characterize crystalline (L)-malate salt of Compound 1.

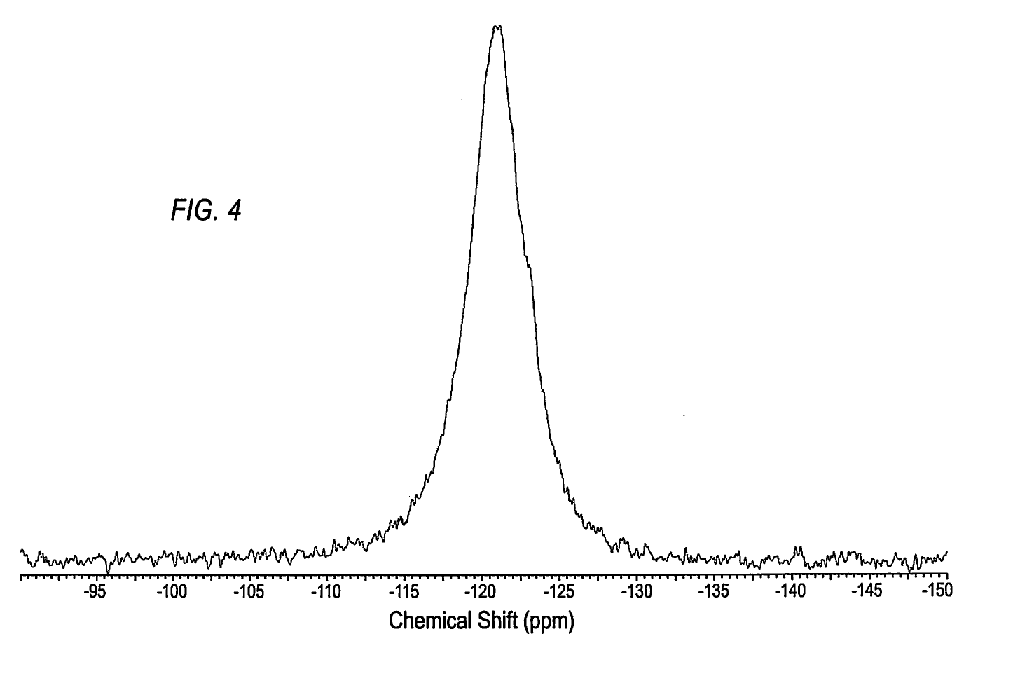

FIG. 4 shows the solid state ¹⁹F NMR spectrum of the crystalline (L)-malate salt of Compound 1. The spectrum shows a peak at −121.6, −120.8, and −118.0 ppm, ±0.2 ppm.

### Thermal Characterization Measurements

### Thermal Gravimetric Analysis (TGA)

The TGA measurements were performed in a TA Instruments™ model Q500 or 2950, employing an open pan setup. The sample (about 10-30 mg) was placed in a previously tared platinum pan. The weight of the sample was measured accurately and recorded to a thousand of a milligram. The furnace was purged with nitrogen gas at 100 mL/min. Data were collected between room temperature and 300° C. at 10° C./min heating rate.

Differential Scanning calorimetry (DSC) Analysis

DSC measurements were performed in a TA Instruments™ models Q2000, 1000, or 2920, employing an open pan setup. The sample (about 2-6 mg) was weighed in an aluminum pan, accurately recorded to a hundredth of a milligram, and transferred to the DSC. The instrument was purged with nitrogen gas at 50 mL/min. Data were collected between room temperature and 300° C. at a 10° C./min heating rate. The plot was made with the endothermic peaks pointing down.

### (L)-Malate Salt of Compound 1

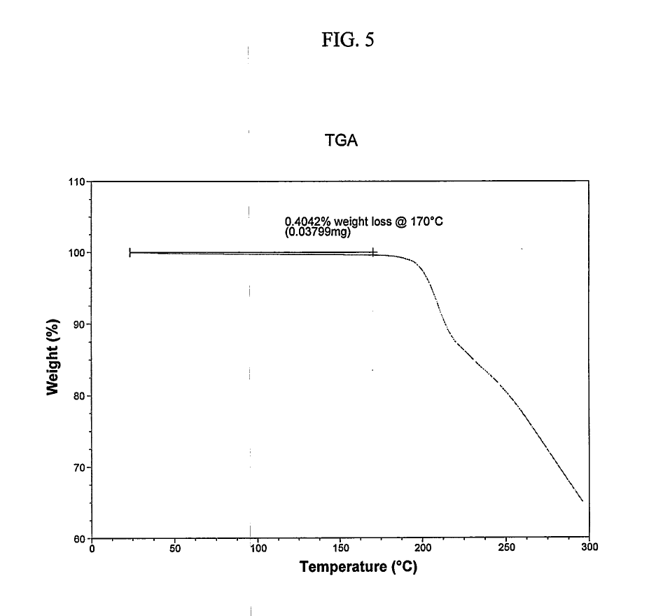

FIG. 5 shows the TGA thermogram for the crystalline (L)-malate salt of Compound 1, which shows a weight loss of approximately 0.4 weight percent at a temperature of 170° C.

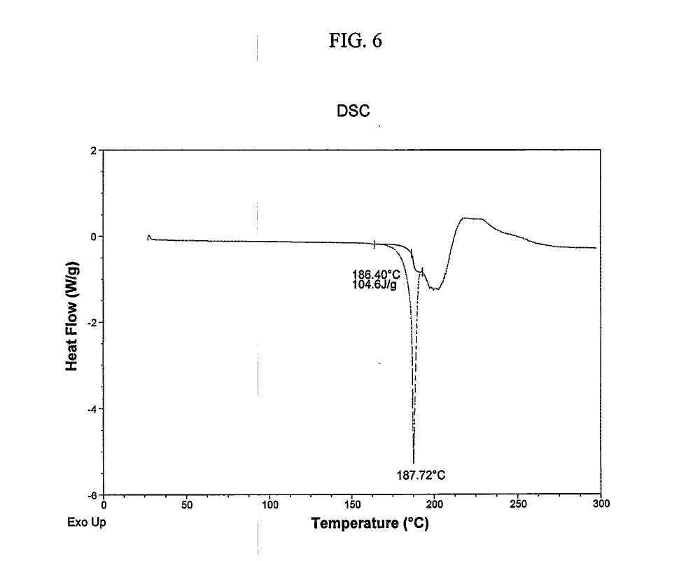

FIG. 6 shows the DSC thermogram for the crystalline (L)-malate salt of Compound 1, which showed a melting point of approximately 187° C.

### Moisture Vapor Isotherm Measurements

Moisture sorption isotherms were collected in a VTI SGA-100 Symmetric Vapor Analyzer using approximately 10 mg of sample. The sample was dried at 60° C. until the loss rate of less than or equal to 0.0005 weight percent per minute was obtained for 10 minutes. The sample was tested at 25° C. and a relative humidity (RH) of 3, 4, 5, 15, 25, 35, 45, 50, 65, 75, 85, and 95 percent. Equilibration at each RH was reached when the rate of less than or equal to 0.0003 weight percent per minute for 35 minutes was achieved, or at a maximum of 600 minutes.

### (L)-Malate Salt of Compound 1

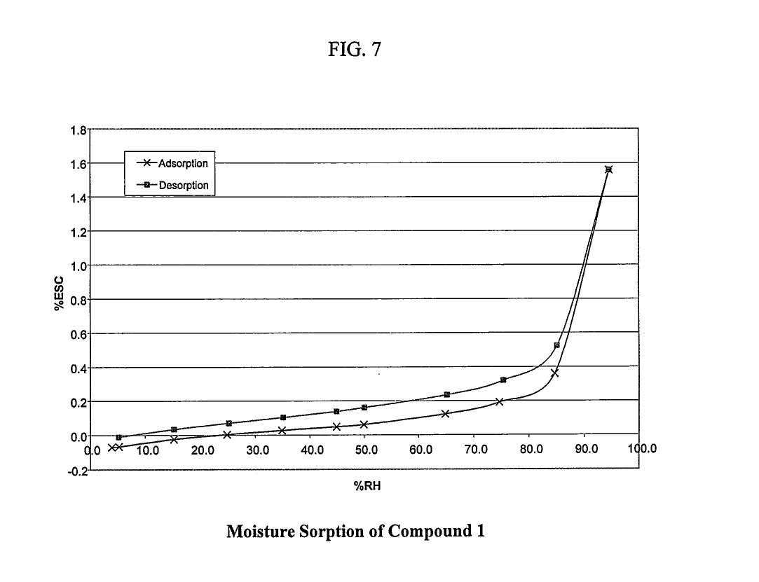

FIG. 7 shows the moisture vapor isotherm of the crystalline (L)-malate salt of Compound 1.

### General Administration

The description below, as it applies to the administration of the compound of Formula I, I(a), or Compound 1, is also applicable to the modes of adiminatration of GEM as well as the administration of all other anti-cancer agents described herein. In certain other embodiments, administration is by the oral route. Administration of the compound of Formula I, I(a), or compound 1, or a pharmaceutically acceptable salt thereof, in pure form or in an appropriate pharmaceutical composition, can be carried out via any of the accepted modes of administration or agents for serving similar utilities. Administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as tablets, suppositories, pills, soft elastic and hard gelatin dosages (which can be in capsules or tablets), powders, solutions, suspensions, or aerosols, or the like, specifically in unit dosage forms suitable for simple administration of precise dosages.

The compositions will include a conventional pharmaceutical carrier or excipient and a compound of Formula I, I(a), or Compound 1 as the/an active agent, and also may include carriers, adjuvants, and the like.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the compound of Formula I, I(a), or Compound 1 may also contain minor amounts of auxiliary substances such as wetting or emulsifying agents, pH buffering agents, antioxidants, and the like, for example, citric acid, sorbitan monolaurate, triethanolamine oleate, butylalted hydroxytoluene, and the like.

The choice of formulation depends on various factors, such as the mode of drug administration (e.g., for oral administration, formulations in the form of tablets, pills or capsules) and the bioavailability of the drug substance. Recently, pharmaceutical formulations have been developed especially for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propyleneglycol, polyethyleneglycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions, and by the use of surfactants.

One specific route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the compound of Formula I, I(a), or Compound 1 is admixed with at least one inert customary excipient (or carrier), such as sodium citrate or dicalcium phosphate, or (a) fillers or extenders, for example, starches, lactose, sucrose, glucose, mannitol, and silicic acid; (b) binders, for example, cellulose derivatives, starch, alignates, gelatin, polyvinylpyrrolidone, sucrose, and gum acacia; (c) humectants, for example, glycerol; (d) disintegrating agents, for example, agar-agar, calcium carbonate, potato or tapioca starch, alginic acid, croscarmellose sodium, complex silicates, and sodium carbonate; (e) solution retarders, for example paraffin; (f) absorption accelerators, for example, quaternary ammonium compounds; (g) wetting agents, for example, cetyl alcohol, glycerol monostearate, magnesium stearate, and the like; (h) adsorbents, for example, kaolin and bentonite; and (i) lubricants, for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms, as described above, can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., the compound of Formula I, I(a), or Compound 1, or a pharmaceutically acceptable salt thereof, and optional pharmaceutical adjuvants in a carrier, for example, water, saline, aqueous dextrose, glycerol, ethanol, and the like; solubilizing agents and emulsifiers, for example, ethyl alcohol, isopropyl alcohol, ethyl carbonate, ethyl acetate, benzyl alcohol, benzyl benzoate, propyleneglycol, 1,3-butyleneglycol, and dimethylformamide; oils, for example, cottonseed oil, groundnut oil, corn germ oil, olive oil, castor oil, and sesame oil; glycerol; tetrahydrofurfuryl alcohol; polyethyleneglycols; fatty acid esters of sorbitan; or mixtures of these substances, and the like, to thereby form a solution or suspension.

In addition to the active compounds, suspensions may contain suspending agents, for example, ethoxylated isostearyl alcohols, polyoxyethylene sorbitol, sorbitan esters, microcrystalline cellulose, aluminum metahydroxide, bentonite, agar-agar and tragacanth, or mixtures of these substances, and the like.

Compositions for rectal administration are, for example, suppositories that can be prepared by mixing the compound of Formula I, I(a), or Compound 1 with suitable non-irritating excipients or carriers, such as cocoa butter, polyethyleneglycol, or a suppository wax, which are solid at ordinary temperatures but liquid at body temperature and therefore melt while in a suitable body cavity and release the active component therein.

Dosage forms for topical administration of the compound of Formula I, I(a), and Compound 1 include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this dislcosure.

Compressed gases may be used to disperse the compound of Formula I, I(a), or Compound 1 in aerosol form. Inert gases suitable for this purpose are nitrogen, carbon dioxide, and the like.

Generally, depending on the intended mode of administration, the pharmaceutically acceptable compositions will contain about 1 percent to about 99 percent by weight of a compound(s) of Formula I, I(a), or Compound 1, or a pharmaceutically acceptable salt thereof, and 99 percent to 1 percent by weight of a suitable pharmaceutical excipient. In one example, the composition will be between about 5 percent and about 75 percent by weight of a compound(s) of Formula I, I(a), or Compound 1, or a pharmaceutically acceptable salt thereof, with the rest being suitable pharmaceutical excipients.

Actual methods of preparing such dosage forms are known, or will be apparent, to those skilled in this art. For example, see Remington's Pharmaceutical Sciences, 18th Ed., (Mack Publishing Company, Easton, Pa., 1990). The composition to be administered will contain a therapeutically effective amount of a compound of Formula I, I(a), or Compound 1, or a pharmaceutically acceptable salt thereof, for treatment of a disease-state in accordance with the teachings of this disclosure.

The compounds of this disclosure, or their pharmaceutically acceptable salts or solvates, are administered in a therapeutically effective amount which will vary depending upon a variety of factors, including the activity of the specific compound employed, the metabolic stability and length of action of the compound, the age, body weight, general health, sex, and diet of the patient, the mode and time of administration, the rate of excretion, the drug combination, the severity of the particular disease-states, and the host undergoing therapy. The compound of Formula I, I(a), or Compound 1 can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors, including the requirements of the patient, the severity of the condition being treated, and the pharmacological activity of the compound being used. The determination of optimum dosages for a particular patient is well known to one of ordinary skill in the art.

If formulated as a fixed dose, such combination products employ the compound of Formula I, I(a), or Compound 1 within the dosage range described above and the other pharmaceutically active agent(s) within its approved dosage range. Compounds of Formula I, I(a), or Compound 1 may alternatively be used sequentially with known pharmaceutically acceptable agent(s) when a combination formulation is inappropriate.

In another embodiment, the compound of Formula I, I(a), or Compound 1 can be administered to the patient concurrently with GEM. In another embodiment, the compound of Formula I, I(a), or Compound 1 is administered to the patient after the administration of GEM. In another embodiment, GEM is administered to the patient after the administration of the compound of Formula I, I(a), or Compound 1.

In one embodiment, GEM and the compound of Formula I, I(a), or Compound 1 are each administered to the patient, in any of the modes of administration as described above, for a period of time ranging from about 4 months to about 10 months. In another embodiment, GEM and the compound of Formula I, I(a), or Compound 1 are each administered to the patient, in any of the modes of administration as described above, for about 4 months. In another embodiment, GEM and the compound of Formula I, I(a), or Compound 1 are each administered to the patient, in any of the modes of administration as described above, for about 5 months. In another embodiment, GEM and the compound of Formula I, I(a), or Compound 1 are each administered to the patient, in any of the modes of administration as described above, for about 6 months. In another embodiment, GEM and the compound of Formula I, I(a), or Compound 1 are each administered to the patient, in any of the modes of administration as described above, for about 7 months. In another embodiment, GEM and the compound of Formula I, I(a), or Compound 1 are each administered to the patient, in any of the modes of administration as described above, for about 8 months. In another embodiment, GEM and the compound of Formula I, I(a), or Compound 1 are each administered to the patient, in any of the modes of administration as described above, for about 9 months. In another embodiment, GEM and the compound of Formula I, I(a), or Compound 1 are each administered to the patient, in any of the modes of administration as described above, for about 10 months. In another embodiment, GEM and the compound of Formula I, I(a), or Compound 1 are each administered to the patient, in any of the modes of administration as described above, for more than 10 months.

In another embodiment, the administration of the compound of Formula I, I(a), or Compound 1 and GEM includes a rest phase, wherein, during the rest phase, neither the compound of Formula I, I(a), or Compound 1 nor GEM is administered to the patient. The rest phase is can range from about 2 weeks to about 12 weeks. In another embodiment, the rest phase can range from about 3 weeks to about 6 weeks. In another embodiment, the rest phase is about 4 weeks in duration.

In another embodiment, the compound of Formula I, I(a), or Compound 1 and GEM can each be administered daily, in any of the modes of administration described above, as 10-300 mg dosages each (for example, in capsules or tablets). In another embodiment, the compound of Formula I, I(a), or Compound 1 and GEM can each be administered daily, in any of the modes of administration described above, as 10-200 mg dosages each (for example, in capsules or tablets). In another embodiment, the compound of Formula I, I(a), or Compound 1 and GEM can each be administered daily, in any of the modes of administration described above, as 20-150 mg dosages each (for example, in capsules or tablets). In another embodiment, the compound of Formula I, I(a), or Compound 1 and GEM can each be administered daily, in any of the modes of administration described above, as 25-100 mg dosages each (for example, in capsules or tablets).

For purposes of this disclosure, for all examples that are disclosed herein that refer to the compound of Formula I, I(a), or Compound 1 or GEM in dosage amounts in milligrams (mg), it is to be read as mg of the parcticular compound, and this dosage amount can be administered in any form, including tablet and capsule form. The examples of capsule or tablet forms within the parenthesis after the dosage amounts listed above are non-limiting examples of how the dosages can be administered. For example, in the above embodiments, GEM can be administered in modes other than capsules or tablets.

In non-limiting examples in all of the above embodiments, the compound of Formula I, I(a), or Compound 1 and GEM can each be administered in 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, and 300 mg dosages (which can, for example, be in capsules or tablets).

### Utility

Compounds of Formula I, I(a), or Compound 1 have been tested using the methods described in the Biological Examples and have been determined to be c-Met inhibitors. As such, compounds of Formula I, I(a), and Compound 1 are useful for treating diseases, particularly cancers such as stomach cancer, esophageal carcinoma, kidney cancer, liver cancer, ovarian carcinoma, cervical carcinoma, large bowel cancer, small bowel cancer, brain cancer (including astrocytic tumor, which includes astocytoma, glioblastoma, giant cell glioblastoma, gliosarcoma, and glioblastoma with oligodendroglial components), lung cancer (including non-small cell lung cancer), bone cancer, prostate cancer, pancreatic cancer, skin cancer, bone cancer, lymphoma, solid tumors, lymphoma, solid tumors, Hodgkin's disease, non-Hodgkin's lymphoma, and thyroid cancer (including medullary thyroid cancer). Suitable in vitro assays for measuring c-Met activity and the inhibition thereof by compounds are known in the art. Suitable in vivo models for cancer are also known to those of ordinary skill in the art. Following the examples disclosed herein, as well as that disclosed in the art, a person of ordinary skill in the art can determine what combinations of a compound of Formula I, I(a), or Compound 1 and anti-cancer agents would be effective for treating cancer.

### Preaparation of Compound 1

Compounds of this invention can be made by the synthetic procedures described below. These procdures are merely illustrative of some methods by which the compounds of Formula I, I(a), or Compound 1 can be synthesized, and various modifications to these procudures may be made. The starting materials and the intermediates of the reaction may be isolated and purified, if desired, using conventional techniques, including, but not limited to, filtration, distillation, crystallization, chromatography, and the like. Such materials may be characterized using conventional means, including physical constants and spectral data.

The disclosure is further illustrated by the following examples, which are not to be construed as limiting the disclosure in scope or spirit to the specific procedures described in them.

### Preparation of N-(4-{[6,7-bis(methyloxy)quinolin-4-yl]oxy}phenyl)-N-(4-fluorophenyl)cyclopropane-1,1-dicarboxamide and the (L)-malate salt thereof

The synthetic route used for the preparation of N-(4-{[6,7-bis(methyloxy)quinolin-4-yl]oxy}phenyl)-N′-(4-fluorophenyl)cyclopropane-1,1-dicarboxamide (Compound 1) and the (L)-malate salt thereof is depicted in Scheme 1.

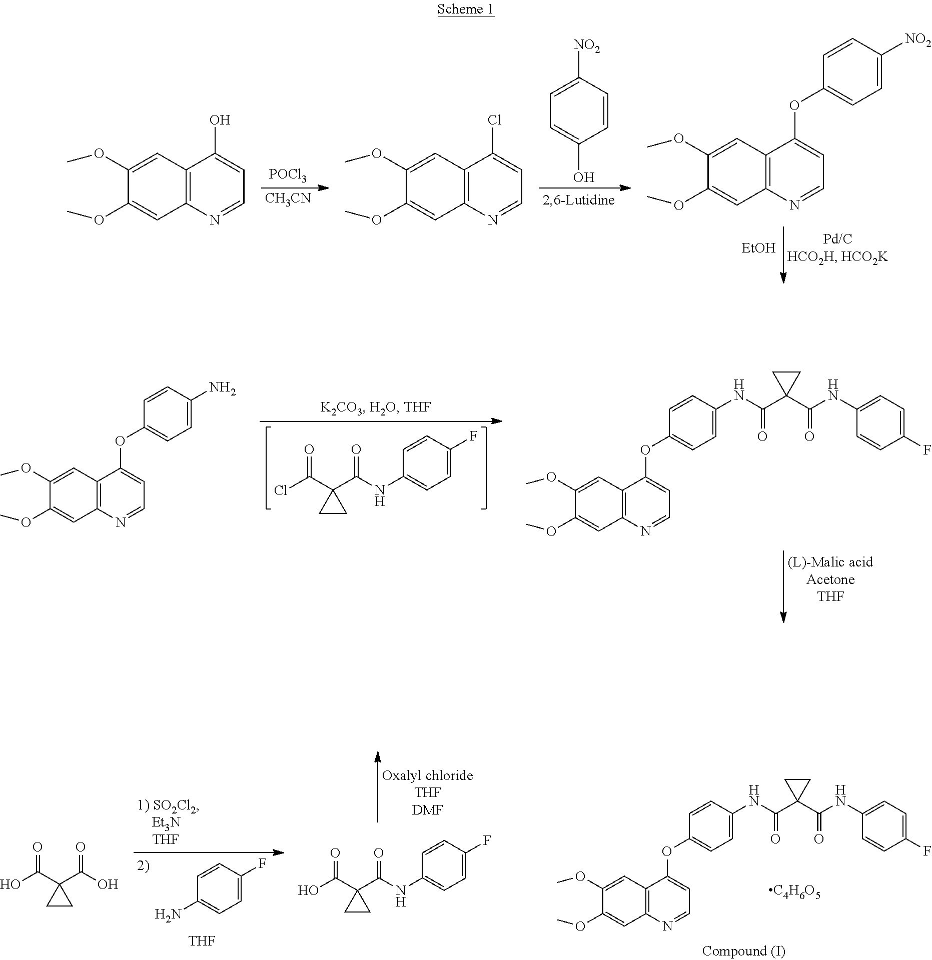

### Preparation of 4-Chloro-6,7-dimethoxy-quinoline

A reactor was charged sequentially with 6,7-dimethoxy-quinoline 4 of (1 1, 10.0 kg) and acetonitrile (64.0 L). The resulting mixture was heated to approximately 65° C., and phosphorus oxychloride (POCl₃, 50.0 kg) was added. The temperature of the reaction mixture was subsequently raised to approximately 80° C. The reaction was deemed complete (approximately 9.0 hours) when less than 2 percent of the starting material remained (in process high-performance liquid chromotography [HPLC] analysis). The reaction mixture was cooled to approximately 10° C. and then quenched into a chilled solution of dichloromethane (DCM, 238.0 kg), 30% NH₄OH (135.0 kg), and ice (440.0 kg). The resulting mixture was warmed to approximately 14° C., and the phases were separated. The organic phase was washed with water (40.0 kg) and concentrated by vacuum distillation with the removal of solvent (approximately 190.0 kg). Methyl-t-butyl ether (MTBE, 50.0 kg) was added to the batch, and the mixture was cooled to approximately 10° C., during which time the product crystallized out. The solids were recovered by centrifugation, washed with n-heptane (20.0 kg), and dried at approximately 40° C. to afford the title compound (8.0 kg).

### Preparation of 6,7-Dimethyl-4-(4 nitro-phenoxy)-quinoline

A reactor was sequentially charged with 4-chloro-6,7-dimethoxy-quinoline (8.0 kg), 4 nitrophenol (7.0 kg), 4 dimethylaminopyridine (0.9 kg), and 2,6-lutidine (40.0 kg). The reactor contents were heated to approximately 147° C. When the reaction was complete (less than percent starting material remaining as determined by in process HPLC analysis; approximately 20 hours), the reactor contents were allowed to cool to approximately 25° C. Methanol (26.0 kg) was added, followed by potassium carbonate (3.0 kg) dissolved in water (50.0 kg). The reactor contents were stirred for approximately 2 hours. The resulting solid precipitate was filtered, washed with water (67.0 kg), and dried at 25° C. for approximately 12 hours to afford the title compound (4.0 kg).

### Preparation of 4-(6,7-Dimethoxy-quinoline-4-yloxy)-phenylamine

A solution containing potassium formate (5.0 kg), formic acid (3.0 kg), and water (16.0 kg) was added to a mixture of 6,7-dimethoxy-4-(4-nitro-phenoxy)-quinoline (4.0 kg), 10 percent palladium on carbon (50 percent water wet, 0.4 kg) in tetrahydrofuran (40.0 kg) that had been heated to approximately 60° C. The addition was carried out such that the temperature of the reaction mixture remained approximately 60° C. When the reaction was deemed complete as determined using in-process HPLC analysis (less than 2 percent starting material remaining, typically 1.5 hours), the reactor contents were filtered. The filtrate was concentrated by vacuum distillation at approximately 35° C. to half of its original volume, which resulted in the precipitation of the product. The product was recovered by filtration, washed with water (12.0 kg), and dried under vacuum at approximately 50° C. to afford the title compound (3.0 kg; 97 percent AUC).

### Preparation of 1-(4-Fluoro-phenylcarbamoyl)-cyclopropanecarboxylic acid

Triethylamine (8.0 kg) was added to a cooled (approximately 4° C.) solution of commercially available cyclopropane-1,1-dicarboxylic acid (2 1, 10.0 kg) in THF (63.0 kg) at a rate such that the batch temperature did not exceed 10° C. The solution was stirred for approximately 30 minutes, and then thionyl chloride (9.0 kg) was added, keeping the batch temperature below 10° C. When the addition was complete, a solution of 4-fluoroaniline (9.0 kg) in THF (25.0 kg) was added at a rate such that the batch temperature did not exceed 10° C. The mixture was stirred for approximately 4 hours and then diluted with isopropyl acetate (87.0 kg). This solution was washed sequentially with aqueous sodium hydroxide (2.0 kg dissolved in 50.0 L of water), water (40.0 L), and aqueous sodium chloride (10.0 kg dissolved in 40.0 L of water). The organic solution was concentrated by vacuum distillation, followed by the addition of heptane, which resulted in the precipitation of solid. The solid was recovered by centrifugation and then dried at approximately 35° C. under vacuum to afford the title compound (10.0 kg).

### Preparation of 1-(4-Fluoro-phenylcarbamoyl)-cyclopropanecarbonyl chloride

Oxalyl chloride (1.0 kg) was added to a solution of 1-(4-fluoro-phenylcarbamoyl)-cyclopropanecarboxylic acid (2.0 kg) in a mixture of THF (11 kg) and N, N-dimethylformamide (DMF; 0.02 kg) at a rate such that the batch temperature did not exceed 30° C. This solution was used in the next step without further processing.

### Preparation of N-(4-{[6,7-bis(methyloxy)quinolin-4-yl]oxy}phenyl)-N′-(4-fluorophenyncyclopropane-1,1-dicarboxamide

The solution from the previous step containing 1-(4-fluoro-phenylcarbamoyl)-cyclopropanecarbonyl chloride was added to a mixture of 4-(6,7-dimethoxy-quinoline-4-yloxy)-phenylamine (3.0 kg) and potassium carbonate (4.0 kg) in THF (27.0 kg) and water (13.0 kg) at a rate such that the batch temperature did not exceed 30° C. When the reaction was complete (typically in about 10 minutes), water (74.0 kg) was added. The mixture was stirred at 15 to 30° C. for approximately 10 hours, which resulted in the precipitation of the product. The product was recovered by filtration, washed with a pre-made solution of THF (11.0 kg) and water (24.0 kg), and dried at approximately 65° C. under vacuum for approximately 12 hours to afford the title compound (free base, 5.0 kg). ¹H NMR (400 MHz, d₆-DMSO): δ 10.2 (s, 1H), 10.05 (s, 1H), 8.4 (s, 1H), 7.8 (m, 2H), 7.65 (m, 2H), 7.5 (s, 1H), 7.35 (s, 1H), 7.25 (m, 2H), 7.15 (m, 2H), 6.4 (s, 1H), 4.0 (d, 6H), 1.5 (s, 4H). LC/MS: M+H=502.

### Preparation of N-(4-{[6,7-bis(methyloxy)quinolin-4-yl]oxy}phenyl)-N′-(4-fluorophenyl)cyclopropane-1,1-dicarboxamide, (L)-malate salt

A solution of L-malic acid (2.0 kg) in water (2.0 kg) was added to a solution of Cyclopropane-1,1-dicarboxylic acid [4-(6,7-dimethoxy-quinoline-4-yloxy)-phenyl]-amide (4-fluoro-phenyl)-amide free base (15, 5.0 kg) in ethanol, maintaining a batch temperature of approximately 25° C. Carbon (0.5 kg) and thiol silica (0.1 kg) were then added, and the resulting mixture was heated to approximately 78° C., at which point water (6.0 kg) was added. The reaction mixture was then filtered, followed by the addition of isopropanol (38.0 kg), and was allowed to cool to approximately 25° C. The product was recovered by filtration, washed with isopropanol (20.0 kg), and dried at approximately 65° C. to afford the title compound (5.0 kg).

### Biological Examples

### Establishment of Primary Tumor Xenografts

Samples of human pancreatic adenocarcinomas were obtained. Within 60 minutes following surgical resection, tumors were suspended in sterile RPMI medium 1640 (available from Sigma-Aldrich®), mechanically dissociated using scissors, then minced with a sterile scalpel blade over ice to yield 2×2 mm pieces. The tumor pieces were washed with serum-free PBS before implantation. Eight-week-old male NOD/SCID mice were anesthetized using an i.p. injection of 100 mg/kg ketamine and 5 mg/kg xylazine. NOD/SCID mice are publically available from Charles River Laboratories International in Wilmington, Mass. A 5-mm incision was then made in the skin overlying the mid-abdomen, and three pieces of tumor were implanted subcutaneously. The skin incision was closed with absorbable suture. The mice were monitored weekly for tumor growth for 16 to 20 weeks. After establishment of xenografts, studies were performed on passage 2 tumors.

### Preparation of Single Cell Suspensions of Tumor Cells

Xenograft tumors or primary human tumors were cut up into small pieces with scissors, and then minced completely using sterile scalpel blades. To obtain single cell suspensions, the resultant minced tumor pieces were mixed with ultra-pure collagenase IV (Worthington Biochemical, Lakewood, N.J.) in medium 199 (200 units of collagenase per ml) and allowed to incubate at 37° C. for 1 to 1.5 hours for enzymatic dissociation. The specimens were further mechanically dissociated every 10 minutes by pipetting with a 10-ml pipette. At the end of the incubation, cells were filtered through a 40-μm nylon mesh, washed with HBSS/20% FBS, and then washed twice with HBSS. HBSS is Hank's Balanced Salt Solution, and FBS is Fetal Bovine Serum, both of which can be obtained from Thermo Fisher Scientific Inc. The preparation from a 1 cm³ xenograft tumor typically resulted in 20-30 million cancer cells.

### Tumorsphere Cultures

Following fluorescence-activated call sorting (FACS), single cells suspensions were washed twice using serum-free HBSS. Cells were resuspended in culture media containing 1 percent N2 supplement (Gibco, Carlsbad, Calif.), 2% B27 supplement (Gibco), 1% Antibiotic-Antimycotic (Gibco), 20 ng/ml human bFGF-2 (Invitrogen, Carlsbad, Calif.), and 20 ng/ml EGF (Gibco). The cells were then plated in 6-well ultralow attachment plates (Corning, Corning, N.Y.). Plates were analyzed for sphere forming colonies (tumorspheres) and were quantified using an inverted microscope (Leica, Allendale, N.J.). For subsequent passaging of spheres or generation of single cell suspensions, tumorspheres were collected, dissociated with 0.05 percent trypsin, and sieved through a 40 μm strainer. Dissociated cells were washed with serum-free HBSS for cell sorting or cultured in ultralow attachment plates as needed for subsequent experimentation.

Implantation of Pancreatic Cancer Cells into NOD/SCID Mice

Sorted cells or bulk pancreatic tumor cells were washed with serum free HBSS and suspended in serum free-RPMI1640/Matrigel mixture (1:1 volume), followed by injection into the subcutaneous tissue of the right and left mid-abdominal area using a 27-gauge needle. In separate experiments, mice were anesthetized with an i.p. injection of 100 mg/kg ketamine and 5 mg/kg xylazine, and a small median laparotomy was performed. Five thousand sorted cells or 1-5×10⁵ bulk tumor cells resuspended in PBS/Matrigel mixture (1:1) in a volume of 100 □l were injected into the tail of the pancreas using a 30-gauge needle. PBS stands for phosphate buffered saline, which can be obtained from Thermo Fisher Scientific Inc. Six mice were inoculated per test group. Animals then underwent autopsy, and tumor growth was accessed after 4 to 8 weeks. Tissues were fixed in formaldehyde and were examined histologically or dissociated to generate single cell suspensions for further study.

### Immunoblot Analysis

Immunoblot analysis was done according to Zhang (2004) using antibodies directed against phospho-c-Met (Santa Cruz Biotechnology, Santa Cruz, Calif.), c-Met (Upstate, Temecula, Calif.), and phospho-ERK (Cell Signaling Technology, Beverly, Mass.), all at a dilution of 1:1000. After analysis, the blots were stripped, washed, and re-probed with a β-actin antibody (Sigma, St Louis, Mo.) to serve as an additional loading control. Protein expression was quantified using a Kodak Gel Documentation System (model 1D 3.6).

### Measurement of Apoptosis

Cells were labelled with AnnexinV (FITC) and propidium iodide (PI) according to the manufacturer's instructions (BD Biosciences, Palo Alto, Calif.) to quantify the percentage of cells undergoing apoptosis. Annexin V and PI staining (fluorescence intensity) were assessed by fluorescence-activated call sorting (FACS) (Becton Dickinson, San Jose, Calif.). The percentage of apoptotic cells was expressed as a percentage of the total population. Experiments were performed in triplicate, and each was repeated three times.

### Bioluminescent Imaging

Bioluminescent imaging of implanted orthotopic tumors in mice was performed using a Xenogen IVIS 200 Imaging System (Xenogen Biosciences, Cranbury, N.J.) as previously described (Charafe-Jauffret et al., 2009). Prior to imaging, animals were anesthetized in an acrylic chamber with a 1.5 percent isofluorane/air mixture and injected i.p. with 40 mg/mL of luciferin potassium salt in PBS at a dose of 150 mg/kg body weight. To validate the findings with bioluminescent imaging, mice were euthanized with carbon dioxide inhalation, and autopsies were performed to assess the extent of primary tumor growth and metastasis.

The Synergistic Combination of GEM and Compound 1 Decreases Tumor Growth and Reduces Cancer Stem Cells in Vivo for Cancer Stem Cells that are Injected under the Skin (Ectopic Xenograft)

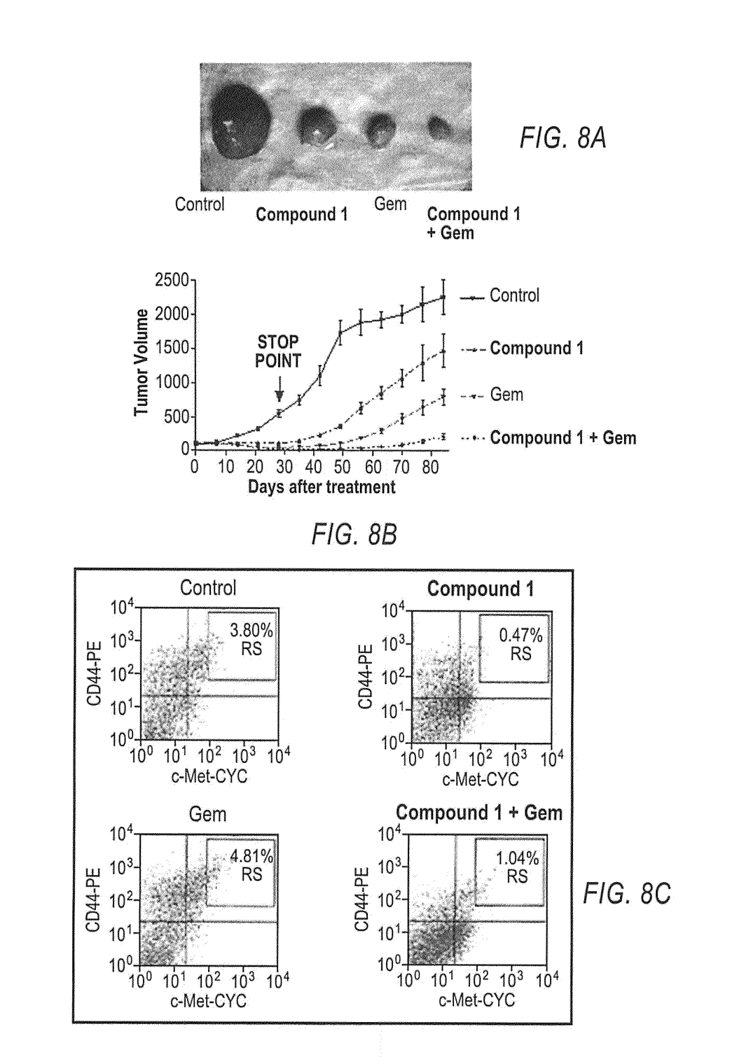

The effect of Compound 1 (30 mg/kg/day) on three separate human pancreatic adenocarcinomas established in NOD/SCID mice was tested. The effects of Compound 1 with the effects of GEM (100 mg/kg/twice weekly), used alone or in combination, was tested. The combination of Compound 1 and GEM resulted in the synergistic effect of tumor growth inhibition, wherein tumor growth was prevented for up to 8 weeks following cessation of treatment (FIG. 8B). As a note, although GEM alone was able to reduce total cancer cells as shown in FIG. 8, it concomitantly lead to increases in the total number of pancreatic cancer stem cells as shown in Table 1A. The combined treatment of Compound 1 and GEM, however, not only surprisingly prevented the increase in the cancer stem cell population that was observed with GEM treatment alone, but also resulted in a decease in CD44+c-Met+ population by 63±7 percent (Table 1A). These synergistic effects of Compound 1 and GEM in ectopic xenografts are described in more detail below. CD44 and c-Met are both markers on pancreatic cancer stem cells.

TABLE 1A
Effect of Compound 1 and GEM on CD44+c-Met+ and
CD44+CD24+ESA+ cancer stem cell populations in
subcutaneous xenograft tumor.

| | Treatment | | | |
|---|---|---|---|---|
| | | | Com- | Compound 1 + |
| | Control | Gem | pound 1 | GEM |
| % of CD44+c-Met+ | 3.13 ± | 4.34 ± | 0.55 ± | 1.08 ± |
| | 0.77% | 0.68% | 0.20% | 0.17% |

Flow cytometry was performed to isolate CD44+c-Met+ human pancreatic cancer stem cells of each treatment group. Compound 1 treatment significantly depleted CD44+c-Met+ cancer stem cell population.

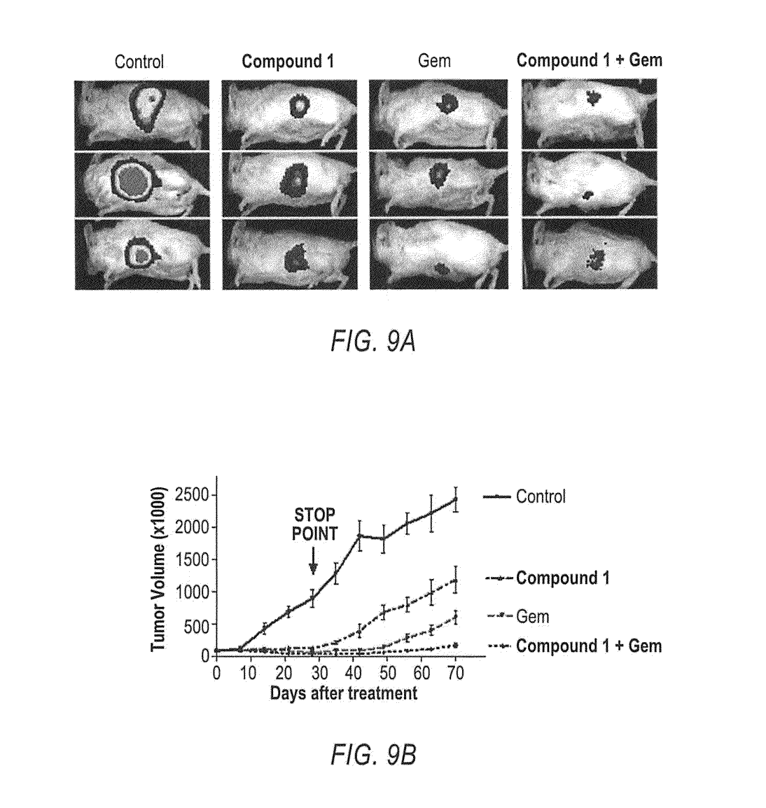

NOD/SCID mice (n=6 per group) were subcutaneously injected with 5×10⁵ pancreatic cancer cells, and when the average tumor volume reached approximately 100 mm³, the mice were either left untreated or treated with Compound 1, GEM, or the combination of the two agents. Subcutaneous tumor volume was assessed weekly. All of the mice tolerated the treatments well, without significant weight change or change in activity. Half of the mice in each group were euthanized 4 weeks after initiation of treatment, and the tumors were dissociated and examined using flow cytometry to determine the percentage of c-Met+/CD44+ cancer stem cells within the tumors. The effect of the different treatment arms on tumor size at 4 weeks is shown in FIGS. 8A and 8B. The remaining mice were kept for an additional 8 weeks following cessation of treatment, and tumor volume was measured weekly. Both Compound 1 and GEM significantly inhibited tumor growth during the 4 week period of treatment (FIG. 8B). Following cessation of treatment, the tumors in the Compound 1 and GEM treatment groups grew more slowly than the untreated tumors. As stated above, the tumors that had been treated with a combination of Compound 1 and GEM had the surprising effect of preventing tumor growth for up to 8 weeks following cessation of treatment (FIG. 8B). GEM treatment alone resulted in an increase in 48±20 percent of the c-Met+CD44+ population. The results showed that treatment with Compound 1 resulted in a significant decrease of 83±3 percent in the percentage of c-Met+/CD44+ cancer stem cells within the treated tumors, suggesting that treatment with Compound 1 targeted a cancer stem cell population within the tumor. Combined treatment with Compound 1 and GEM was not only able to prevent the increase in the cancer stem cell population observed with GEM treatment alone, but also surprisingly resulted in a decease in CD44+c-Met+ population by 63±7 percent. Similar results when measuring the effects of Compound 1, used alone or in combination with GEM, on the CD44+CD24+ESA+ cancer stem cell population are shown in FIG. 9 and Table 1B.

In FIG. 8A, subcutaneous low passage primary pancreatic adenocarcinomas from three different patient tumors (approximately 100 mm² in size) were treated with saline (control), Compound 1 (30 mg/kg/day), GEM (100 mg/kg/twice weekly), and the combination of Compound 1 and GEM for 4 weeks (n=6 animals per group). Tumor size was measured weekly. Three mice of each group were euthanized after 4 weeks treatment for analysis of cancer stem cells, and the other mice were followed to monitor tumor growth for an additional 8 weeks. A representative experiment demonstrating the actual resected tumor of each treatment group. In FIG. 8B, the average tumor size±SEM from three separate tumors is presented. FIG. 8C depicts a graph of CSC percentage in different treatment groups. The percentage of the c-Met+/CD44+ tumorigenic cancer cell population in the total cancer cell population is indicated for each treatment.

### The Synergistic Combination of GEM and Compound 1 Decreases Tumor Growth and Reduces Cancer Stem Cells in Vivo for Cancer Stem Cells Planted in the Pancreas (Orthotopic Implants)

Based on the effects of Compound 1 on subcutaneous tumors established in vivo (Example 8), the effects of Compound 1, used alone or in combination with GEM, was analyzed in primary human pancreatic cancer in which tumors were established as orthotopic implants in the pancreatic tail of NOD/SCID mice. The combination of Compound 1 and GEM resulted in the synergistic effects of tumor growth inhibition, wherein tumor growth was completely prevented for up to 6 weeks following cessation of treatment (FIG. 9B). As a note, as before, although GEM alone was able to reduce total cancer cells as shown in FIG. 9, it concomitantly lead to increases in the total number of pancreatic cancer stem cells, as shown in Table 1B. However, the combined treatment of Compound 1 and GEM not only surprisingly prevented the increase in the cancer stem cell population that was observed with GEM treatment alone, but also resulted in a decease in CD44+CD24+ESA+ population by 1.4±0.47 percent (Table 1B). These synergistic effects of Compound 1 and GEM in Orthotopic Implants are described in more detail below.

TABLE 1B
Effect of Compound 1 and GEM on CD44+c-Met+ and
CD44+CD24+ESA+ cancer stem cell populations in
subcutaneous xenograft tumor.

| | Treatment | | | |
|---|---|---|---|---|
| | | | Com- | Compound 1 + |
| | Control | GEM | pound 1 | GEM |
| % of CD44+CD24+ESA+ | 3.07 ± | 4.3 ± | 0.61 ± | 1.4 ± |
| | 0.74% | 1.61% | 0.18% | 0.47% |

Flow cytometry was performed to isolate CD44+CD24+ESA+ human pancreatic cancer stem cells of each treatment group. Compound 1 treatment significantly depleted CD44+CD24+ESA+ cancer stem cell population.

The same three pancreatic cancers used for the subcutaneous tumor implantation experiments were infected with a lentivirus expressing luciferase to allow imaging in real time, and cells were injected into the pancreatic tail. Once the tumors reached 1-5×10⁵ photons count per second, treatment with Compound 1, GEM, or the combination of both agents was begun as performed in the previous treatment study with established subcutaneous tumors. Treatment with Compound 1 or GEM prevented tumor growth during the 4 week treatment and significantly inhibited tumor growth compared to controls, while the combined treatment with Compound 1 and GEM acted in as synergistic fashion and completely prevented tumor growth for the 6 week period of study after cessation of treatments.

In FIG. 9, the human pancreatic cancer cells infected with a luciferase-expressing lentivirus were directly injected into the pancreatic tail of NOD/SCID mice (n=6 animals per group), and treatment started 2 weeks after injection. Tumor size and volume were measured weekly using Xenogen IVIS 200 imaging system throughout the experiment period. FIG. 9A shows representative bioluminescent images of three of the animals in each group are shown at 4 weeks after treatment, depicting the extent of tumor burden. The summary results from three separate experiments are presented in FIG. 9B.

### Other Embodiments

The foregoing disclosure has been described in some detail by way of illustration and example, for purposes of clarity and understanding. The invention has been described with reference to various specific and preferred embodiments and techniques. It should be understood, however, that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive.

The scope of the invention should be determined not with reference to the above description, but should instead be determined with reference to the following appended claims, along with the full scope of equivalents to which such claims are entitled.

1-11. (canceled)

12. A method for inhibiting tumor growth and reducing CD44+c-Met+ or CD44+CD24+ESA+ cancer stem cell population in a human adenocarcinoma, wherein the method comprises administering to a patient in need of the treatment a therapeutically effective amount of Compound 1: 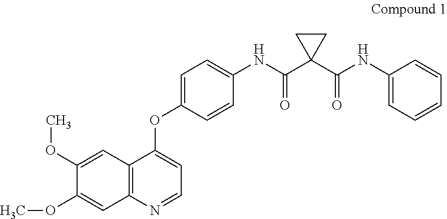 [SMILES: COc1cc2nccc(Oc3ccc(NC(=O)C4(C(=O)Nc5ccccc5)CC4)cc3)c2cc1OC]
or a pharmaceutically acceptable salt thereof, in combination with gemcitabine (GEM), or a pharmaceutically acceptable salt thereof, and optionally radiation.

13. The method according to claim 12, wherein Compound 1 is the malate salt.

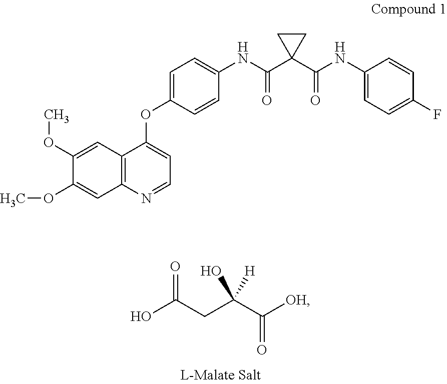 [SMILES: COc1cc2nccc(Oc3ccc(NC(=O)C4(C(=O)Nc5ccc(F)cc5)CC4)cc3)c2cc1OC ; O=C(O)C[C@@H](O)C(=O)O]

14. The method according to claim 13, wherein the salt is crystalline.

15. The method according to claim 12, wherein Compound 1, or a pharmaceutically acceptable salt thereof, is administered as a pharmaceutical composition further comprising a pharmaceutically acceptable carrier, excipient, or diluent.

16. A method of treating pancreatic cancer in a patient by inhibiting tumor growth and reducing CD44+c-Met+ or CD44+CD24+ESA+ cancer stem cell population, wherein the method comprises administering to a patient in need of the treatment Compound 1: 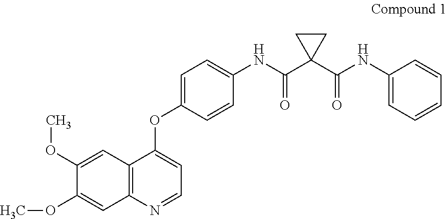 [SMILES: COc1cc2nccc(Oc3ccc(NC(=O)C4(C(=O)Nc5ccccc5)CC4)cc3)c2cc1OC]
or a pharmaceutically acceptable salt thereof, in combination with gemcitabine (GEM), or a pharmaceutically acceptable salt thereof, and optionally radiation.
